(19) United States
(12) Patent Application Publication
(10) Pub. No.: US 2020/0033366 A1
(43) Pub. Date: Jan. 30, 2020

(54) SYSTEMS AND METHODS FOR METABOLOME ANALYSIS

(71) Applicant: 10X GENOMICS, INC., Pleasanton, CA (US)

(72) Inventor: Luigi Jhon Alvarado Martinez, Pleasanton, CA (US)

(21) Appl. No.: 16/434,099

(22) Filed: Jun. 6, 2019

Related U.S. Application Data
(60) Provisional application No. 62/756,495, filed on Nov. 6, 2018.
(60) Provisional application No. 62/711,351, filed on Jul. 27, 2018.

(51) Int. Cl. G01N 33/68; C12Q 1/6886; G01N 33/574

(52) U.S. Cl. CPC G01N 33/6893; C12Q 1/6886; G01N 33/57484; G16B 20/00; G01N 2800/52; G01N 2800/60; G01N 2560/00; G01N 33/6848

(57) ABSTRACT

The disclosure provides systems and methods for detecting and analyzing the metabolome of a cell. Such methods and systems can include or make use of barcoding of nucleic acid molecules and their sequencing.

### CROSS-REFERENCE

This application claims the benefit U.S. Provisional Patent Application No. 62/756,495, filed Nov. 6, 2018, and U.S. Provisional Patent Application No. 62/711,351, filed Jul. 27, 2018, each of which is entirely incorporated herein by reference.

### BACKGROUND

A sample may be processed for various purposes, such as identification of a type of moiety within the sample. The sample may be a biological sample. Biological samples may be processed, such as for detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Biological samples in partitions may be subjected to various processes, such as chemical processes or physical processes. Samples in partitions may be subjected to heating or cooling, or chemical reactions, such as to yield species that may be qualitatively or quantitatively processed.

Partitioning of cells and/or cellular materials can be useful for analyzing nucleic acid molecules that are endogenous to the cell.

### SUMMARY

While partitioning of cells and/or cellular materials may be useful in the analysis of endogenous nucleic acids, analysis of other cellular components, such that analysis can be linked to a particular cell, remains challenging. Such analyses include the analysis of cellular metabolites. Profiling the array of metabolites of a cell, the metabolome, can be useful in evaluating a host of cellular characteristics, including cellular processes, function and viability. Accordingly, recognized herein is a need for improved systems and methods for detecting and analyzing cellular compounds. In particular, systems and methods described herein are useful in detecting and analyzing the metabolome of a cell.

An aspect of the present disclosure provides a method for processing or analyzing a metabolite from a cell. The method comprises: (a) generating a partition comprising (i) the metabolite, (ii) a single bead comprising a plurality of nucleic acid cell barcode molecules each comprising a cell barcode sequence and a capture sequence, which cell barcode sequence uniquely corresponds to the cell, and (iii) a molecular complex that is capable of coupling to the metabolite, where the molecular complex comprises a molecular complex capture sequence and a molecular complex identifier sequence, where the molecular complex capture sequence is inaccessible to the capture sequence in the absence of the metabolite coupled to the molecular complex; (b) in the partition, providing conditions sufficient for the metabolite to couple to the molecular complex to (i) render the molecular complex capture sequence accessible to the capture sequence of a given nucleic acid cell barcode molecule of the plurality of nucleic acid cell barcode molecules and (ii) permit the capture sequence to couple to the molecular complex capture sequence; and (c) with the capture sequence coupled to the molecular complex capture sequence, using the given nucleic acid cell barcode molecule and the molecular complex to synthesize a nucleic acid molecule comprising (1) a first nucleic acid sequence corresponding to the molecular complex identifier sequence, and (2) a second nucleic acid sequence corresponding to the cell barcode sequence, where the first nucleic acid sequence and the second nucleic acid sequence permit the metabolite to be identified as corresponding to the cell. In some cases, a corresponding sequence may be a complementary sequence.

In some embodiments, the method further comprises sequencing at least a portion of the nucleic acid molecule or a derivative thereof, to identify the molecular complex identifier sequence and the cell barcode sequence. In some embodiments, the method further comprises using the molecular complex identifier sequence and the cell barcode sequence to identify the metabolite as originating from the cell. In some embodiments, the molecular complex is a riboswitch. In some embodiments, the partition comprises the cell. In some embodiments, the method further comprises, after (a), releasing the metabolite from the cell.

In some embodiments, the method further comprises, prior to (c), releasing the nucleic acid cell barcode molecule from the single bead. In some embodiments, the nucleic acid cell barcode molecule is released from the single bead upon exposure to a chemical stimulus in the partition. In some embodiments, (c) is performed in the partition. In some embodiments, the method further comprises releasing or recovering the nucleic acid molecule or a derivative thereof from the partition. In some embodiments, in (c), the nucleic acid molecule is synthesized using one or more of a nucleic acid amplification reaction, a reverse transcription reaction and a template switching reaction.

In some embodiments, the method further comprises subjecting the nucleic acid molecule to one or more additional reactions. In some embodiments, the one or more additional reactions comprise a primer extension reaction. In some embodiments, the one or more additional reactions comprise addition of one or more functional sequences to the nucleic acid molecule, where the one or more functional sequences are configured to permit attachment of the nucleic acid molecule or a derivative thereof to a flow cell of a sequencer.

In some embodiments, the single bead is a gel bead. In some embodiments, while the nucleic acid molecule is synthesized, the nucleic acid cell barcode molecule is attached to the single bead. In some embodiments, the partition comprises an additional molecular complex that is capable of coupling to an additional metabolite that is different than the metabolite. In some embodiments, the single bead comprises an additional plurality of nucleic acid cell barcode molecules each comprising the cell barcode sequence and an additional capture sequence capable of coupling to an additional molecular complex capture sequence of the additional molecular complex when the additional metabolite is coupled to the additional molecular complex. In some embodiments, the method further comprises in the partition, permitting the additional metabolite to couple to the additional molecular complex to (iii) render the additional molecular complex capture sequence accessible to the additional capture sequence of a given additional nucleic acid cell barcode molecule of the additional plurality of nucleic acid cell barcode molecules and (iv) permit the additional capture sequence to couple to the additional molecular complex capture sequence; and with the additional capture sequence coupled to the additional molecular complex capture sequence, using the given additional nucleic acid cell barcode molecule and the additional molecular complex to synthesize an additional nucleic acid molecule comprising (3) a third nucleic acid sequence corresponding to the additional molecular complex identifier sequence, and (4) a fourth additional nucleic acid sequence corresponding to the cell barcode sequence, where the third nucleic acid sequence and the fourth nucleic acid sequence permit the additional metabolite to be identified with the cell.

In some embodiments, the plurality of nucleic acid cell barcode molecules comprises at least 100,000 nucleic acid cell barcode molecules. In some embodiments, the partition is a droplet among a plurality of droplets. In some embodiments, the partition is a well among a plurality of wells. In some embodiments, the metabolite is selected from the group consisting of adenosine triphosphate (ATP), adenosine diphosphate (ADP), adenosine monophosphate (AMP), guanosine triphosphate (GTP), guanosine monophosphate (GMP), ribose, glucose, mannose, glycerol, phosphatidyl choline, phosphoryl choline, glyceryl phosphoryl choline, phosphatidyl serine, phosphatidyl ethanolamine, diglyceride, nicotinamide adenine dinucleotide phosphate, nicotinamide adenine dinucleotide, glycine, glutamine, aspartic acid, citrate, glycerine, acetone, acetoacetic acid and lysine.

In some embodiments, each of the plurality of nucleic acid cell barcode molecules comprises an identifier sequence separate from the cell barcode sequence, and where the identifier sequence is different for each nucleic acid cell barcode molecule of the plurality of nucleic acid cell barcode molecules. In some embodiments, where, in (a), each of the plurality of nucleic acid cell barcode molecules comprises the cell barcode sequence, where the single bead is from a plurality of beads, and where the cell barcode sequence is different from cell barcode sequences of nucleic acid cell barcode molecules of other beads of the plurality of beads.

In some embodiments, where the single bead comprises a plurality of additional nucleic acid cell barcode molecules each comprising the cell barcode sequence and a binding sequence, which binding sequence is capable of coupling to an additional nucleic acid molecule different from the molecular complex. In some embodiments, the additional nucleic acid molecule is selected from the group consisting of a ribonucleic acid molecule of the cell, a deoxyribonucleic acid molecule of the cell, and an editing nucleic acid molecule capable of participating in a gene editing reaction. In some embodiments, the method further comprises, prior to (a), exposing a protein of the cell to an antibody coupled to the additional nucleic acid molecule, where the antibody binds to the protein. In some embodiments, the partition comprises the additional nucleic acid molecule, and the method further comprises, permitting a given additional nucleic acid cell barcode molecule of the additional plurality of nucleic acid cell barcode molecules to bind to the additional nucleic acid molecule via the binding sequence. In some embodiments, the method further comprises using the additional nucleic acid molecule and the given additional nucleic acid cell barcode molecule of the additional plurality of nucleic acid cell barcode molecules to synthesize a reporter nucleic acid molecule comprising (3) a third nucleic acid sequence corresponding to a sequence of the additional nucleic acid molecule, and (4) a fourth nucleic acid sequence corresponding to the cell barcode sequence. In some embodiments, the method further comprises sequencing at least a portion of the reporter nucleic acid molecule or a derivative thereof, to identify the species and as associated with the cell.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual biological particles or analyte carriers;

FIG. 2 shows an example of a microfluidic channel structure for delivering barcode carrying beads to droplets;

FIG. 3 shows an example of a microfluidic channel structure for co-partitioning biological particles or analyte carriers and reagents;

FIG. 4 shows an example of a microfluidic channel structure for the controlled partitioning of beads into discrete droplets;

FIG. 5 shows an example of a microfluidic channel structure for increased droplet generation throughput;

FIG. 6 shows another example of a microfluidic channel structure for increased droplet generation throughput;

FIG. 7A shows a cross-section view of another example of a microfluidic channel structure with a geometric feature for controlled partitioning. FIG. 7B shows a perspective view of the channel structure of FIG. 7A;

FIG. 8 illustrates an example of a barcode carrying bead;

FIG. 9 shows a computer system that is programmed or otherwise configured to implement methods provided herein;

FIG. 10 schematically illustrates an example molecular complex, a riboswitch, and its function upon binding of a metabolite;

FIG. 11A schematically illustrates an example method for detecting and analyzing a metabolite of a cell;

FIG. 11B schematically illustrates an example scheme for generating barcoded sequencing constructs derived from a molecular complex;

FIG. 11C schematically illustrates an example scheme for adding additional sequences to barcoded sequencing constructs; and

FIG. 12 schematically illustrates an example bead comprising nucleic acid cell barcode molecules that can be used to detect and analyze a plurality of different types of species.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The terms “biological particle” or “analyte carrier,” as used herein, generally refers to a discrete biological system derived from a biological sample. The biological particle or analyte carrier may comprise, or carry therein, an analyte (e.g., biological analyte) of interest. In some embodiments, the analyte carrier is itself the analyte of interest. The biological particle or analyte carrier may be a macromolecule. The biological particle or analyte carrier may be a small molecule. The biological particle or analyte carrier may be a virus. The biological particle or analyte carrier may be a cell or derivative of a cell. The biological particle or analyte carrier may be an organelle. The biological particle or analyte carrier may be a rare cell from a population of cells. The biological particle or analyte carrier may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological or analyte carrier particle may be a constituent of a cell. The biological particle or analyte carrier may be or may include DNA, RNA, organelles, proteins, or any combination thereof. The biological particle or analyte carrier may be or may include a matrix (e.g., a gel or polymer matrix) comprising a cell or one or more constituents from a cell (e.g., cell bead), such as DNA, RNA, organelles, proteins, or any combination thereof, from the cell. The biological particle or analyte carrier may be obtained from a tissue of a subject. The biological particle or analyte carrier may be a hardened cell. Such hardened cell may or may not include a cell wall or cell membrane. The biological particle or analyte carrier may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix. As used herein, the terms “biological particle” and “analyte carrier” may be used interchangeably.

The term “macromolecular constituent,” as used herein, generally refers to a macromolecule contained within or from a biological particle or analyte carrier. The macromolecular constituent may comprise a nucleic acid. In some cases, the biological particle or analyte carrier may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### Analysis of Cellular Metabolomes

Provided are systems and methods for analyzing the metabolome of a cell. A metabolite of a cell can be partitioned into a partition such that its cellular origin can be identified. The partition can also include molecular complexes that can uniquely couple with the metabolite and have nucleic acid sequences that identify the molecular complex and, thus, the metabolite. Such sequences can be determined and used to identify the metabolite as originating from the cell.

In an aspect, the disclosure provides a method for processing or analyzing a metabolite from a cell. The method comprises: (a) generating a partition comprising (i) the metabolite, (ii) a single bead (e.g., a single gel bead) comprising a plurality of nucleic acid cell barcode molecules each comprising a cell barcode sequence and a capture sequence, which cell barcode sequence uniquely corresponds to the cell, and (iii) a molecular complex that is capable of coupling to the metabolite, where the molecular complex comprises a molecular complex capture sequence and a molecular complex identifier sequence, where the molecular complex capture sequence is inaccessible to the capture sequence in the absence of the metabolite coupled to the molecular complex; (b) in the partition, providing conditions sufficient for the metabolite to couple to the molecular complex to (i) render the molecular complex capture sequence accessible to the capture sequence of a given nucleic acid cell barcode molecule of the plurality of nucleic acid cell barcode molecules and (ii) permit the capture sequence to couple to the molecular complex capture sequence; and (c) with the capture sequence coupled to the molecular complex capture sequence, using the given nucleic acid cell barcode molecule and the molecular complex to synthesize a nucleic acid molecule comprising (1) a first nucleic acid sequence corresponding to the molecular complex identifier sequence, and (2) a second nucleic acid sequence corresponding to the cell barcode sequence, where the first nucleic acid sequence and the second nucleic acid sequence permit the metabolite to be identified with the cell.

In some cases, the plurality of nucleic acid cell barcode molecules comprises at least about 1,000 nucleic acid cell barcode molecules, at least about 5,000 nucleic acid cell barcode molecules, at least about 10,000 nucleic acid cell barcode molecules, at least about 50,000 nucleic acid cell barcode molecules, at least about 100,000 nucleic acid cell barcode molecules, at least about 500,000 nucleic acid cell barcode molecules, at least about 1,000,000 nucleic acid cell barcode molecules, at least about 5,000,000 nucleic acid cell barcode molecules, at least about 10,000,000 nucleic acid cell barcode molecules, at least about 100,000,000 nucleic acid cell barcode molecules, at least about 1,000,000,000 nucleic acid cell barcode molecules, or more. In some cases, the nucleic acid cell barcode molecules comprise the same cell barcode sequence.

Each of the plurality of nucleic acid cell barcode molecules can include an identifier sequence separate from the cell barcode sequence, where the identifier sequence is different for each nucleic acid cell barcode molecule of the plurality of nucleic acid cell barcode molecules. In some cases, such an identifier sequence is a unique molecular identification sequence (UMI) as described elsewhere herein. As described elsewhere herein, UMI sequences can uniquely identify a particular nucleic acid molecule that is barcoded, which may be identifying particular nucleic acid molecules that are analyzed, counting particular nucleic acid molecules that are analyzed, etc. Furthermore, in some cases, including (a), each of the plurality of nucleic acid cell barcode molecules can comprise the cell barcode sequence and the single bead can be from a plurality of beads, such as a population of barcoded beads as described elsewhere herein. Each of the cell barcode sequences can be different from cell barcode sequences of nucleic acid barcode molecules of other beads of the plurality of beads. Where this is the case, a population of barcoded beads, with each bead comprising a different cell barcode sequence can be obtained.

The method can process or analyze any suitable metabolite from the cell. Non-limiting examples of such metabolites include adenosine triphosphate (ATP), adenosine diphosphate (ADP), adenosine monophosphate (AMP), guanosine triphosphate (GTP), guanosine monophosphate (GMP), ribose, glucose, mannose, glycerol, phosphatidyl choline, phosphoryl choline, glyceryl phosphoryl choline, phosphatidyl serine, phosphatidyl ethanolamine, diglyceride, nicotinamide adenine dinucleotide phosphate, nicotinamide adenine dinucleotide, glycine, glutamine, aspartic acid, citrate, glycerine, acetone, acetoacetic acid and lysine.

In general, the molecular complex comprises, at least in part, a nucleic acid component that provides the capture sequence and molecular complex identifier sequence. In some cases, though, the molecular complex comprises a plurality of nucleic acid components, with each of the molecular complex capture sequence and the molecular complex identifier sequence on different nucleic acid components of the molecular complex. The molecular complex identifier sequence can be determined and used to identify the metabolite and the molecular complex capture sequence can be used to aid in generating barcoded molecules corresponding to the molecular complex (e.g., via its molecular complex identifier sequence).

An example of a molecular complex is a riboswitch. A riboswitch can refer to a regulatory segment of a nucleic acid molecule (e.g., a ribonucleic acid molecule (RNA), a messenger RNA, etc.) that can bind a species (including small molecules, metabolites, etc.), resulting in a change in production of proteins encoded by nucleic acid molecules in the cell. Additional details regarding riboswitches are provided in Ruff & Strobel, R N A, 2014 November; 20(11): 1775-88 and Butler et al., Chem Biol. 2011 Mar. 25; 18(3): 293-298, which are both herein entirely incorporated by reference in their entireties for all purposes. A particular riboswitch may uniquely bind to a particular species (such as a particular metabolite) and may also have a unique sequence among other riboswitches. Such a riboswitch identifier sequence can be determined and used to determine binding of the particular species. Moreover, when the species binds to its respective riboswitch, the riboswitch can change its secondary structure and/or tertiary structure such that one or more sequences of the riboswitch, inaccessible in a species-free state, become accessible in a species-bound state. Such a sequence can be used as a capture sequence that can bind a nucleic acid barcode molecule and, via one or more reactions (e.g., one or more primer extension reactions), add a complementary sequence corresponding to the riboswitch (including the riboswitch identifier sequence) to the nucleic acid barcode molecule.

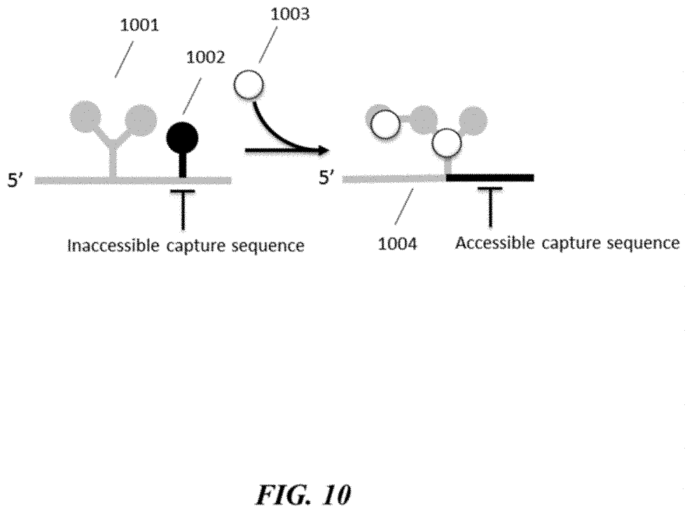

An example riboswitch in species-free and species-bound conformations is schematically depicted in FIG. 10. As shown, riboswitch 1001 comprises an inaccessible capture sequence 1002 when it is not bound to its respective species 1003 (e.g., metabolite) that binds to the riboswitch. In its inaccessible conformation, the capture sequence 1002 cannot bind with other oligonucleotides. When the species 1003 binds to the riboswitch 1001, the capture sequence 1002 becomes accessible. As shown in the example, the riboswitch also includes a riboswitch identifier sequence 1004 that is 5′ to the accessible capture sequence. A barcode nucleic acid molecule can bind to the accessible capture sequence and be extended to add a sequence complementary to the riboswitch identifier sequence 1004 to the nucleic acid barcode molecule.

In some cases, a riboswitch may comprise an aptamer sequence in addition to a capture sequence. The aptamer sequence can engage with the capture sequence when not bound to a ligand, and disengage from the capture sequence when bound to the ligand. The aptamer sequence can also provide one or more binding sites for a ligand such as a metabolite. In some examples, the aptamer sequence and capture sequence are configured in 5′ to 3′ order (e.g., 5′-aptamer sequence-capture sequence-3′). In other examples, they are configured in an opposite configuration (e.g., 5′-capture sequence-aptamer sequence-3′). Moreover, the aptamer sequence may comprise the riboswitch's identifier sequence.

A riboswitch may comprise any suitable number of nucleotides. For example, a riboswitch may comprise at least about 2, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000 or more nucleotides. In some examples, a riboswitch may comprise at most about 1000, 950, 900, 850, 800, 750, 700, 650, 600, 550, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 20, 15, 10, 5 or less nucleotides. Moreover, an aptamer sequence of a riboswitch may comprise at least about 2, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000 or more nucleotides. In some examples, an aptamer sequence of riboswitch may comprise at most about 1000, 950, 900, 850, 800, 750, 700, 650, 600, 550, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 20, 15, 10, 5 or less nucleotides. Furthermore, a capture sequence of a riboswitch may comprise at least about 2, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000 or more nucleotides. In some examples, a capture sequence of riboswitch may comprise at most about 1000, 950, 900, 850, 800, 750, 700, 650, 600, 550, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 20, 15, 10, 5 or less nucleotides.

Additionally, in some cases, the partition comprises the cell (which can be intact) and, after the partition is generated in (a), the method can also include releasing the metabolite from the cell. Release can be performed via any suitable strategy including cell lysis. In such cases, the partition may comprise a cell lysis reagent. In some cases, the partition may comprise a cell bead comprising the cell or at least a portion of its cellular material and including the metabolite. Cell beads and useful applications of them are described in U.S. patent application Ser. No. 15/887,947, which is entirely herein incorporated by reference in its entirety for all purposes. Where cell beads are used, release of the metabolite from the cell may be performed prior to the generation of the partition in (a). In some cases, such as when wells are implemented in completing the method, release of a metabolite from a cell may occur prior to partitioning, such that the wells implemented in partitions contain cells or cellular extracts comprising the metabolite.

Prior to synthesizing the nucleic acid molecule, in (c), the nucleic acid cell barcode molecule can be released from the single bead, such as upon exposure of the single bead to a chemical stimulus in the partition as described elsewhere herein. In some cases, though, the nucleic acid cell barcode molecule is attached to the single bead, such that the synthesized nucleic acid molecule is coupled to the single bead.

Moreover, in (c), the nucleic acid molecule can be synthesized using one or more of nucleic acid amplification reaction, a reverse transcription and a template switching reaction as described elsewhere herein. Any suitable type of nucleic acid amplification can be used for synthesis, including amplification-based barcoding methods including any described elsewhere herein and those described in U.S. Patent Publication No. 2014/0378349, U.S. Patent Publication No. 2016/0257984, and U.S. Patent Publication No. 2015/0376609, which are entirely herein incorporated by reference for all purposes.

The method may also include subjecting the nucleic acid molecule to one or more additional reactions, which may take place in the partition and/or which may take place once the nucleic acid molecule or a derivative of the nucleic acid molecule (e.g., a subsequent nucleic acid molecule that includes at least a portion of the nucleic acid molecule or its complement, etc.) is released or recovered from the partition. Accordingly, the method may further comprise releasing or recovering the nucleic acid molecule from the partition, such as where synthesis of the nucleic acid molecule is performed as in (c), in the partition. Where the partition is a droplet among a plurality of droplets (e.g. droplets of an emulsion as described elsewhere herein), removal or recovery of the nucleic acid molecule or derivative thereof can be achieved by droplet disruption, such as the breaking of an emulsion in which the droplet is situated. Where the partition is a well amount a plurality of wells, the nucleic acid molecule or derivative thereof can be removed from the well, such as with standard fluid transport techniques, like pipetting or other forced fluid flow.

In some cases, the one or more additional reactions may comprise a primer extension reaction or a plurality of primer extension reactions. Such reactions include nucleic acid amplification reactions, like polymerase chain reaction (PCR). Primer extension reactions can be used to add additional sequences to the nucleic acid molecule or derivative thereof via priming of primer binding sites on the nucleic acid molecule or derivative thereof and further extension of the nucleic acid molecule, using the primer and/or a sequence associated with the primer as a template. Primer extension reactions can also be employed to increase the number of copies of nucleic acid molecule or derivative thereof. Any number of primer extension reactions can be implemented depending on the particular construct to be used for analysis.

In some cases, the one or more additional reactions may comprise a ligation reaction (or a ligation reaction in combination with one or more primer extension reactions). Via the action of a ligase, additional sequences can be appended onto the nucleic molecule or derivative thereof.

Furthermore, the one or more additional reactions can add one or more functional sequences to the nucleic acid molecules, which can render the nucleic acid molecule or derivative thereof suitable for nucleic acid sequencer and sequencing analysis. For example, the one or more functional sequences may be configured to permit hybridization of a sequencing primer to nucleic acid molecule or derivative thereof, may be configured as a sample index, or may be configured to permit attachment of the nucleic acid molecule or derivative thereof to a flow cell of sequencer (e.g., an Illumina sequencer or other type of sequencer in which oligonucleotides for sequencing are immobilized).

Moreover, the method can further comprise sequencing at least a portion of the nucleic acid molecule or a derivative thereof, to identify the molecular complex identifier sequence and the cell barcode sequence. The molecular complex identifier sequence can be determined and used to back-determine the particular metabolite bound to the molecular complex. As bound molecular complexes are those that can interact with nucleic acid cell barcode molecules, the molecular complex identifier sequence can uniquely identify particular metabolites present in the partition (and, thus, the underlying cell). Additionally, the cell barcode sequence can be used to identify a particular partition (and thus its contents, which include cellular material from a particular cell) and, thus, the underlying cell from which the metabolite is obtained. Sequencing may be performed using any suitable platform, including next-generation sequencing platforms, such as Illumina.

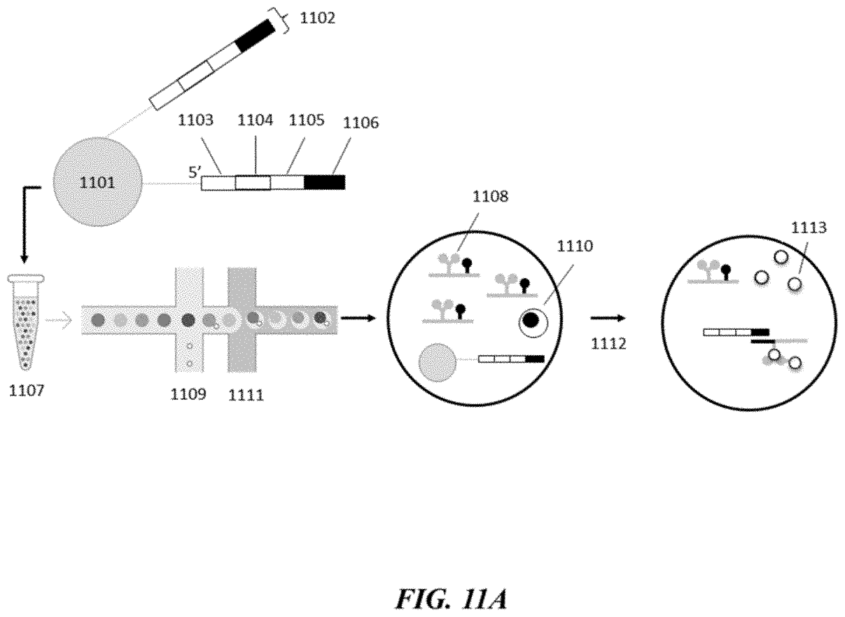

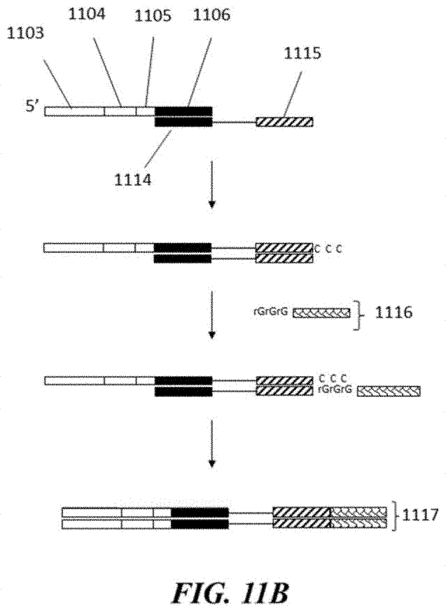

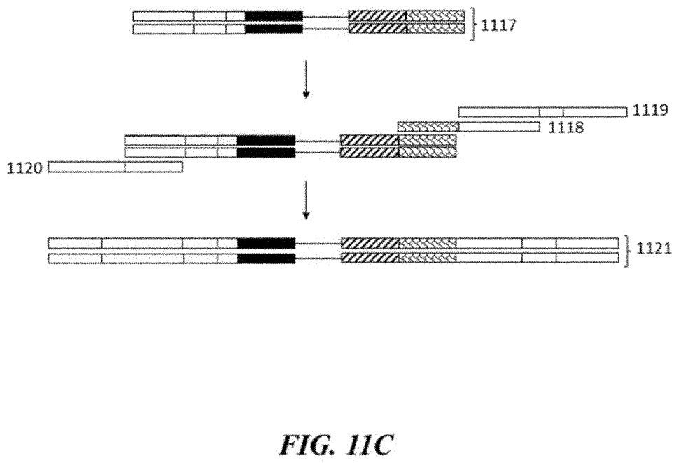

An example method for processing or analyzing a metabolite from a cell is schematically depicted in FIGS. 11A-C. With reference to FIG. 11A, a barcoded bead 1101, among a plurality of barcoded beads 1107, comprises a plurality of nucleic acid cell barcode molecules 1102. Each of the nucleic acid cell barcode molecules 1102 comprises a functional sequence 1103 that permits attachment of the nucleic acid cell barcode molecules 1102 to the flow cell (or other component) of a sequencer, a cell barcode sequence 1104 and a functional sequence 1105 that permits binding of a sequencing primer to the nucleic acid cell barcode molecules 1102. The nucleic acid cell barcode molecules also comprise a capture sequence 1106 that hybridizes with a molecular complex capture sequence. While not shown in FIG. 11, the nucleic acid cell barcode molecules may also comprise an identifier sequence, such as a UMI.

The barcoded bead 1101 is provided in an aqueous mixture to a double-junction microfluidic device where the beads are mixed at a first junction of the microfluidic device with an aqueous stream 1109 comprising molecular complex 1108 that can bind to a particular metabolite, reagents necessary for barcoding, a cell 1111, a cell lysis reagent(s) and a reagent(s) that are capable of releasing the nucleic acid cell barcode molecules 1102 from the bead. The resulting mixture is then passed to a second junction of the microfluidic device where it is contacted with an immiscible stream (e.g., a stream comprising an oil, such as a fluorinated oil and optionally a surfactant), such that a droplet is generated. The droplet comprises the molecular complex 1108, the barcoded bead 1101, the cell 1110, the cell lysis reagents and the reagents necessary for barcoding. The cell lysis reagents lyse 1112 the cell 1110 such that the contents, including a metabolite, of the cell 1110 are released to the droplet.

The freed metabolite 1113 from the cell 1110 then binds with the molecular complex 1108, which changes the conformation of the molecular complex as in FIG. 10, and renders its molecular complex capture sequence accessible to the capture sequence 1106 of the nucleic acid cell barcode molecules 1102. The nucleic acid cell barcode molecules 1102 are released from the barcoded bead with the aid of respective release reagent(s) (e.g., such as a reducing agent that degrades disulfide bonds of the bead, breaks disulfide bonds between the bead and the nucleic acid cell barcode molecules 1102, etc.). A given nucleic acid cell barcode molecule of the released nucleic acid cell barcode molecules 1102 bind with the molecular complex 1108 for barcoding of the molecular complex identifier sequence of the molecular complex 1108.

Barcoding of the molecular complex identifier sequence adds a sequence complementary to the given nucleic acid cell barcode molecule, as schematically depicted in FIG. 11B. With reference to FIG. 11B, the given nucleic acid cell barcode molecule binds via its capture sequence 1106 to the molecular complex capture sequence 1114. The given nucleic acid cell barcode molecule is extended via the action of a polymerase to add a sequence complementary to a sequence of the molecular complex identifier sequence 1115 of the molecular complex 1108. After extension, a terminal transferase adds three cytosine nucleotides to the extended nucleic acid molecule, which allows the extended nucleic acid molecule to participate in a template switch reaction. A template switch oligo 1116, comprising a functional sequence (e.g., a sequencing primer binding site), then hybridizes with the terminal cytosine nucleotides and the extended nucleic acid molecule is then further extended. The resulting product can then be released from the droplet (e.g., breaking of an emulsion comprising the droplet) and further amplified to generate a double-stranded product 1117.

Additional sequences (e.g., additional functional sequences, sample index sequences, etc.) can be added to the double-stranded nucleic acid product 1117. In some cases, such sequences can be added via additional rounds of primer extension reactions and/or amplification, via binding of templates that comprise the additional sequences. An example is shown schematically in FIG. 11C. With reference to FIG. 11C, nucleic acid molecule 1118 can add a first sequence to a strand of double-stranded product 1117 via hybridization and extension/amplification. This added first sequence can then hybridize with another nucleic acid molecule 1119 to add a second sequence via hybridization and extension/amplification. Any number of additional sequences can be added. Likewise, nucleic acid molecule 1120 can be used to add additional sequences to the other end of the product 1117 via hybridization and extension. The completed product 1121 can then be isolated and sequenced for analysis. Sequencing analysis can determine the cell barcode sequence and molecular complex identifier sequence and, thus, identify the metabolite and as originating from the cell.

While not shown in FIG. 11C, other schemes exist for adding additional sequences, including, for example, ligation. For example, any of nucleic acid molecules 1118, 1119 and 1120 can be added to a strand of product 1117 via ligation. In some cases, shearing of product 1117 may be first completed, prior to ligation.

Also, while the example shown in FIGS. 11A and 11B implements a droplet as a partition, the method shown can be analogously performed in another type of partition such as a well. In such cases, the cell, barcoded bead and all other reagents can be provided to the well for cell lysis and barcoding and the contents removed from wells and processed in bulk after barcoding to add further sequences to barcoded products and/or amplify the products as shown in FIG. 11C.

The method can also be used to process or analyze a plurality of different metabolites. Determination of different metabolites can be used to elucidate the cell's metabolome. Accordingly, the partition can include an additional molecular complex that is capable of coupling to an additional metabolite that is different than the metabolite. In some cases, the single bead comprises an additional plurality of nucleic acid cell barcode molecules that each comprises the cell barcode sequence and an additional capture sequence capable of coupling to an additional molecular complex capture sequence of the additional molecular complex when the additional metabolite is coupled to the additional molecular complex. In some cases, the additional capture sequence is the same sequence as the capture sequence. In other cases, the additional capture is a different capture sequence.

Where an additional molecular complex that binds with the additional metabolite is implemented, the method can further comprise permitting the additional metabolite to couple to the additional molecular complex to (iii) render the additional molecular complex capture sequence accessible to the additional capture sequence of a given additional nucleic acid cell barcode molecule of the additional plurality of nucleic acid cell barcode molecules and (iv) permit the additional capture sequence to couple to the additional molecular complex capture sequence. With the additional capture sequence coupled to the additional molecular complex capture sequence, the given additional nucleic acid cell barcode molecule and the additional molecular complex to can be used to synthesize an additional nucleic acid molecule comprising (3) a third nucleic acid sequence corresponding to the additional molecular complex identifier sequence, and (4) a fourth additional nucleic acid sequence corresponding to the cell barcode sequence. The third nucleic acid sequence and the fourth nucleic acid sequence can permit the additional metabolite to be identified with the cell.

As an example, the example method shown in FIGS. 11A-C can be modified such that a library of different molecular complexes is provided to each droplet. Each synthesized nucleic acid molecule would comprise the cell barcode sequence and a sequence corresponding to a particular molecular complex where its corresponding metabolite was present in the cell. Identification of the molecular complex identifier sequences of the various molecular complexes can then be used to identify an array of metabolites present in the cell. The resulting analysis can then be used to construct a metabolome of the cell.

Metabolite processing and analysis can be combined with other analyses in the partition, with non-limiting examples that include transcript analysis, cell surface feature analysis, cell protein analysis and analysis of cellular gene-editing processes and associated nucleic acid molecules. Suitable methods for multi-analyte analysis in a partition are described in U.S. patent application Ser. No. 15/720,085 and PCT International Application No. PCT/US2017/068320, each of which is herein incorporated by reference in its entirety for all purposes.

In multi-analyte analysis, a barcoded bead used for analysis can comprise barcode molecules that can capture different analytes. Accordingly, the single bead may comprise a plurality of additional nucleic acid cell barcode molecules each comprising the cell barcode sequence and a binding sequence, which binding sequence is capable of coupling to an additional nucleic acid molecule different from the molecular complex (e.g., a ribonucleic acid molecule of the cell (such as messenger ribonucleic acid (mRNA), a deoxyribonucleic acid (such as genomic deoxyribonucleic acid (gDNA)), and an editing nucleic acid molecule capable of participating in a gene editing reaction). The partition can comprise an additional nucleic acid molecule, and the method can further comprising permitting a given additional nucleic acid cell barcode molecule of the additional plurality of nucleic acid barcode molecules to bind to the additional nucleic acid molecule via the binding sequence. For example, the given additional nucleic acid cell barcode molecule may bind to a nucleic acid molecule coupled to an agent (e.g., an antibody) that binds a cellular protein, whether on the cell surface or inside the cell. In such cases, prior to partitioning, the protein can be exposed to an antibody coupled to the additional nucleic acid molecule, where the antibody binds to the protein.

Moreover, the additional nucleic acid molecule and the given additional nucleic acid cell barcode molecule of the additional plurality of nucleic acid cell barcode molecules can be used to synthesize a reporter nucleic acid molecule comprising (3) a third nucleic acid sequence corresponding to a sequence of the additional nucleic acid molecule, and (4) a fourth nucleic acid sequence corresponding to the cell barcode sequence. At least a portion of the reporter nucleic acid molecule or a derivative thereof can be sequenced to identify the additional nucleic acid molecule as originating from the cell. Where the additional nucleic acid molecule is coupled to an agent (e.g., antibody) that binds a cellular protein, barcoded nucleic acid molecules can then be generated from the additional nucleic acid molecule, sequenced and the antibody and its target protein identified as originating from the particular cell.

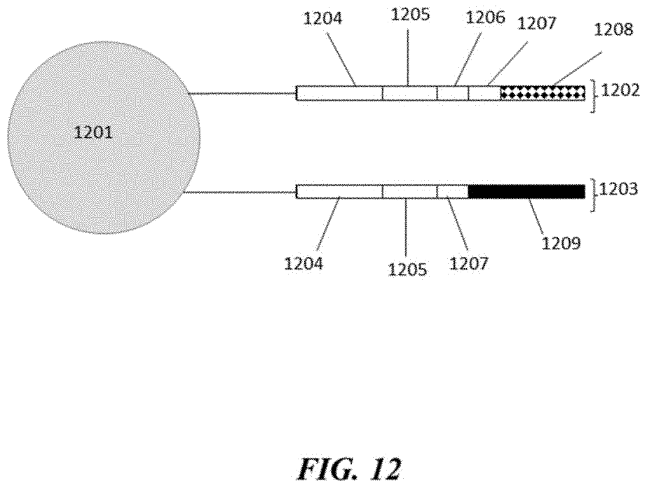

An example barcoded bead that can be provided to a partition for multi-analyte analysis is schematically depicted in FIG. 12. With reference to FIG. 12, the barcoded bead 1201 comprises two nucleic acid cell barcode molecules 1202 and 1203. Nucleic acid cell barcode molecule 1202 comprises a functional sequence 1204 that permits attachment of nucleic acid cell barcode molecule 1202 to a sequencer component; a cell barcode sequence 1205, a UMI sequence 1206, a functional sequence that can bind a sequencing primer 1207 and a capture sequence 1208. The capture sequence 1208 can bind a particular cellular analyte, such as mRNA, gDNA, or a nucleic acid molecule coupled to an agent that binds a cellular protein, etc. Barcoded molecules can be generated in a partition and sequenced (or derivatives sequenced) to identify target molecules as originating from the underlying cell from which cellular material is provided with the barcoded bead 1201 in a partition.

Additionally, nucleic acid cell barcode molecule 1203 comprises sequences 1204, 1205 and 1207 as in nucleic acid cell barcode molecule and also comprises a capture sequence 1209. In some cases, the capture sequence 1209 is the same sequence as capture sequence 1208. In other cases, the capture sequence 1209 is a different sequence as the capture sequence 1208. Capture sequence 1209 can couple with a molecular complex having an accessible binding sequence (e.g., as a result of metabolite binding) and processing/analysis of a metabolite performed as described elsewhere herein.

### Systems and Methods for Sample Compartmentalization

In an aspect, the systems and methods described herein provide for the compartmentalization, depositing, or partitioning of one or more particles (e.g., biological particles or analyte carriers, macromolecular constituents of biological particles or analyte carriers, beads, reagents, etc.) into discrete compartments or partitions (referred to interchangeably herein as partitions), where each partition maintains separation of its own contents from the contents of other partitions. The partition can be a droplet in an emulsion. A partition may comprise one or more other partitions.

A partition may include one or more particles. A partition may include one or more types of particles. For example, a partition of the present disclosure may comprise one or more biological particles or analyte carriers and/or macromolecular constituents thereof. A partition may comprise one or more gel beads. A partition may comprise one or more cell beads. A partition may include a single gel bead, a cell bead, or both a cell bead and single gel bead. A partition may include one or more reagents. Alternatively, a partition may be unoccupied. For example, a partition may not comprise a bead. A cell bead can be a biological particle or analyte carrier and/or one or more of its macromolecular constituents encased inside of a gel or polymer matrix, such as via polymerization of a droplet containing the biological particle or analyte carrier and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual biological particles or analyte carriers, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual particles to discrete partitions may in one non-limiting example be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles or analyte carriers per partition, number of beads per partition, etc.). For example, partition occupancy can be controlled by providing the aqueous stream at a certain concentration and/or flow rate of particles. To generate single biological particle or analyte carrier partitions, the relative flow rates of the immiscible fluids can be selected such that, on average, the partitions may contain less than one biological particle or analyte carrier per partition in order to ensure that those partitions that are occupied are primarily singly occupied. In some cases, partitions among a plurality of partitions may contain at most one biological particle or analyte carrier (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

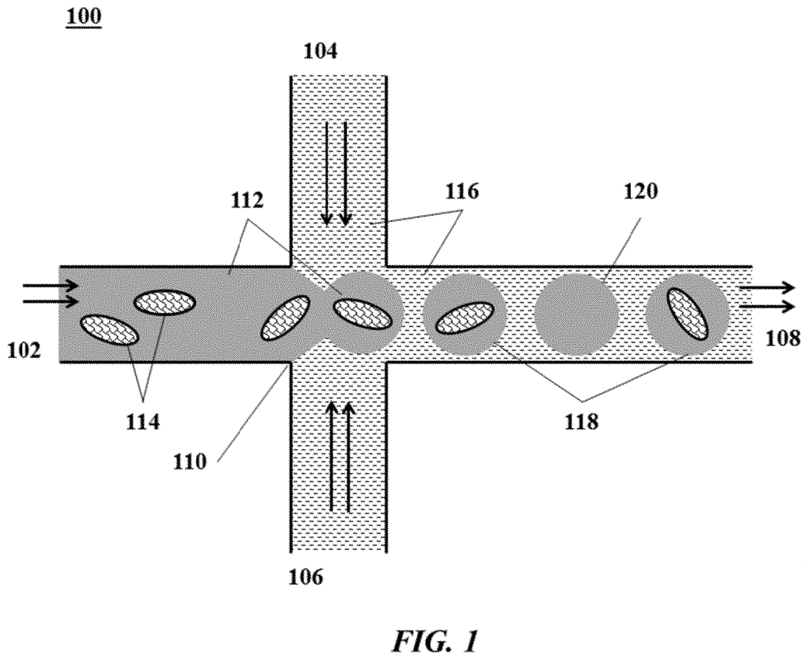

FIG. 1 shows an example of a microfluidic channel structure 100 for partitioning individual biological particles or analyte carriers. The channel structure 100 can include channel segments 102, 104, 106 and 108 communicating at a channel junction 110. In operation, a first aqueous fluid 112 that includes suspended biological particles or analyte carriers (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle or analyte carrier 114 (such as droplets 118). A discrete droplet generated may include more than one individual biological particle or analyte carrier 114 (not shown in FIG. 1). A discrete droplet may contain no biological particle or analyte carrier 114 (such as droplet 120). Each discrete partition may maintain separation of its own contents (e.g., individual biological particle or analyte carrier 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles or analyte carriers, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles or analyte carriers 114, and (2) unoccupied droplets 120, not containing any biological particles or analyte carriers 114. Occupied droplets 118 may comprise singly occupied droplets (having one biological particle or analyte carrier) and multiply occupied droplets (having more than one biological particle or analyte carrier). As described elsewhere herein, in some cases, the majority of occupied partitions can include no more than one biological particle or analyte carrier per occupied partition and some of the generated partitions can be unoccupied (of any biological particle or analyte carrier). In some cases, though, some of the occupied partitions may include more than one biological particle or analyte carrier. In some cases, the partitioning process may be controlled such that fewer than about 25% of the occupied partitions contain more than one biological particle or analyte carrier, and in many cases, fewer than about 20% of the occupied partitions have more than one biological particle or analyte carrier, while in some cases, fewer than about 10% or even fewer than about 5% of the occupied partitions include more than one biological particle or analyte carrier per partition.

In some cases, it may be useful to minimize the creation of excessive numbers of empty partitions, such as to reduce costs and/or increase efficiency. While this minimization may be achieved by providing a sufficient number of biological particles or analyte carriers (e.g., biological particles or analyte carriers 114) at the partitioning junction 110, such as to ensure that at least one biological particle or analyte carrier is encapsulated in a partition, the Poissonian distribution may expectedly increase the number of partitions that include multiple biological particles or analyte carriers. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles or analyte carriers (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

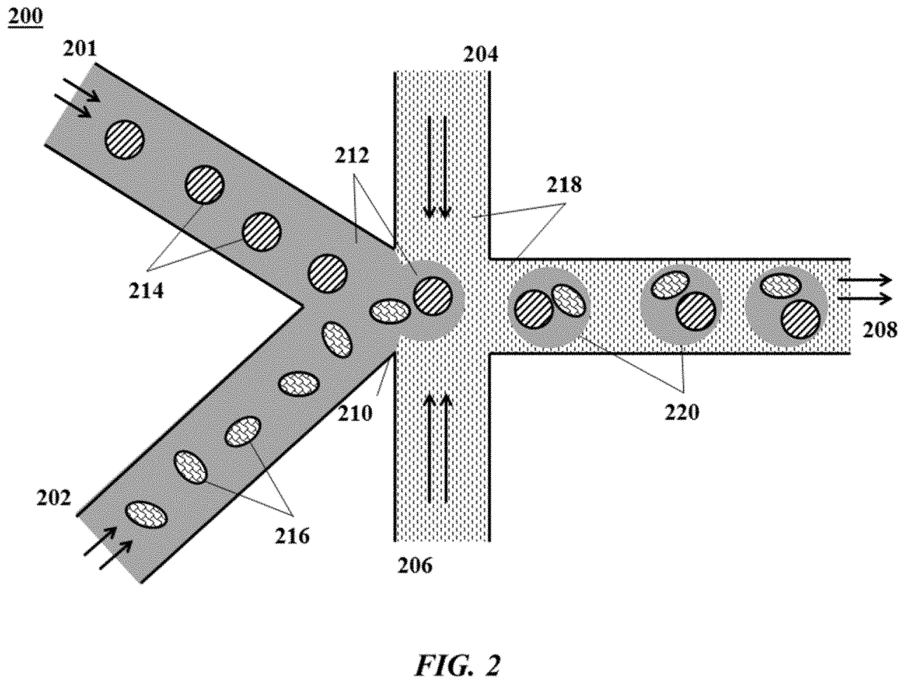

As will be appreciated, the above-described occupancy rates are also applicable to partitions that include both biological particles or analyte carriers and additional reagents, including, but not limited to, microcapsules or beads (e.g., gel beads) carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle or analyte carrier.

In another aspect, in addition to or as an alternative to droplet based partitioning, biological particles or analyte carriers may be encapsulated within a microcapsule that comprises an outer shell, layer or porous matrix in which is entrained one or more individual biological particles or analyte carriers or small groups of biological particles or analyte carriers. The microcapsule may include other reagents. Encapsulation of biological particles or analyte carriers may be performed by a variety of processes. Such processes may combine an aqueous fluid containing the biological particles or analyte carriers with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles or analyte carriers may be performed by a variety of methods. For example, air knife droplet or aerosol generators may be used to dispense droplets of precursor fluids into gelling solutions in order to form microcapsules that include individual biological particles or analyte carriers or small groups of biological particles or analyte carriers. Likewise, membrane based encapsulation systems may be used to generate microcapsules comprising encapsulated biological particles or analyte carriers as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles or analyte carriers 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles or analyte carriers. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca²⁺ ions), can be used as an encapsulation process using the described processes. Likewise, agarose droplets may also be transformed into capsules through temperature based gelling (e.g., upon cooling, etc.).

In some cases, encapsulated biological particles or analyte carriers can be selectively releasable from the microcapsule, such as through passage of time or upon application of a particular stimulus, that degrades the microcapsule sufficiently to allow the biological particles or analyte carriers (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle or analyte carrier can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle or analyte carrier. The polymer or gel may be diffusively permeable to chemical or biochemical reagents. The polymer or gel may be diffusively impermeable to macromolecular constituents of the biological particle or analyte carrier. In this manner, the polymer or gel may act to allow the biological particle or analyte carrier to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle or analyte carrier may be surrounded by polyacrylamide strands linked together by disulfide bridges. In this manner, the biological particle or analyte carrier may be encased inside of or comprise a gel or matrix (e.g., polymer matrix) to form a “cell bead.” A cell bead can contain biological particles or analyte carriers (e.g., a cell) or macromolecular constituents (e.g., RNA, DNA, proteins, etc.) of biological particles or analyte carriers. A cell bead may include a cell or multiple cells, or a derivative of the cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles or analyte carriers and cell beads (and/or droplets or other partitions) containing macromolecular constituents of biological particles or analyte carriers.

Encapsulated biological particles or analyte carriers can provide certain potential advantages of being more storable and more portable than droplet-based partitioned biological particles or analyte carriers. Furthermore, in some cases, it may be useful to allow biological particles or analyte carriers to incubate for a select period of time before analysis, such as in order to characterize changes in such biological particles or analyte carriers over time, either in the presence or absence of different stimuli. In such cases, encapsulation may allow for longer incubation than partitioning in emulsion droplets, although in some cases, droplet partitioned biological particles or analyte carriers may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles or analyte carriers may constitute the partitioning of the biological particles or analyte carriers into which other reagents are co-partitioned. Alternatively or in addition, encapsulated biological particles or analyte carriers may be readily deposited into other partitions (e.g., droplets) as described above.

### Beads

A partition may comprise one or more unique identifiers, such as barcodes. Barcodes may be previously, subsequently or concurrently delivered to the partitions that hold the compartmentalized or partitioned biological particle or analyte carrier. For example, barcodes may be injected into droplets previous to, subsequent to, or concurrently with droplet generation. The delivery of the barcodes to a particular partition allows for the later attribution of the characteristics of the individual biological particle or analyte carrier to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles or analyte carriers 216 along the channel segment 202 into junction 210. The plurality of biological particles or analyte carriers 216 may be sourced from a suspension of biological particles or analyte carriers. For example, the channel segment 202 may be connected to a reservoir comprising an aqueous suspension of biological particles or analyte carriers 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and biological particles or analyte carriers may form a mixture that is directed along another channel to the junction 210 to yield droplets 220. The mixture may provide the beads and biological particles or analyte carriers in an alternating fashion, such that, for example, a droplet comprises a single bead and a single biological particle or analyte carrier.

Beads, biological particles or analyte carriers and droplets may flow along channels at substantially regular flow profiles (e.g., at regular flow rates). Such regular flow profiles may permit a droplet to include a single bead and a single biological particle or analyte carrier. Such regular flow profiles may permit the droplets to have an occupancy (e.g., droplets having beads and biological particles or analyte carriers) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual biological particle or analyte carrier 216. A discrete droplet that is generated may include a barcode or other reagent carrying bead 214. A discrete droplet generated may include both an individual biological particle or analyte carrier and a barcode carrying bead, such as droplets 220. In some instances, a discrete droplet may include more than one individual biological particle or analyte carrier or no biological particle or analyte carrier. In some instances, a discrete droplet may include more than one bead or no bead. A discrete droplet may be unoccupied (e.g., no beads, no biological particles or analyte carriers).

Beneficially, a discrete droplet partitioning a biological particle or analyte carrier and a barcode carrying bead may effectively allow the attribution of the barcode to macromolecular constituents of the biological particle or analyte carrier within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μall), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, lμm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where relatively consistent amounts of reagents within partitions are provided, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

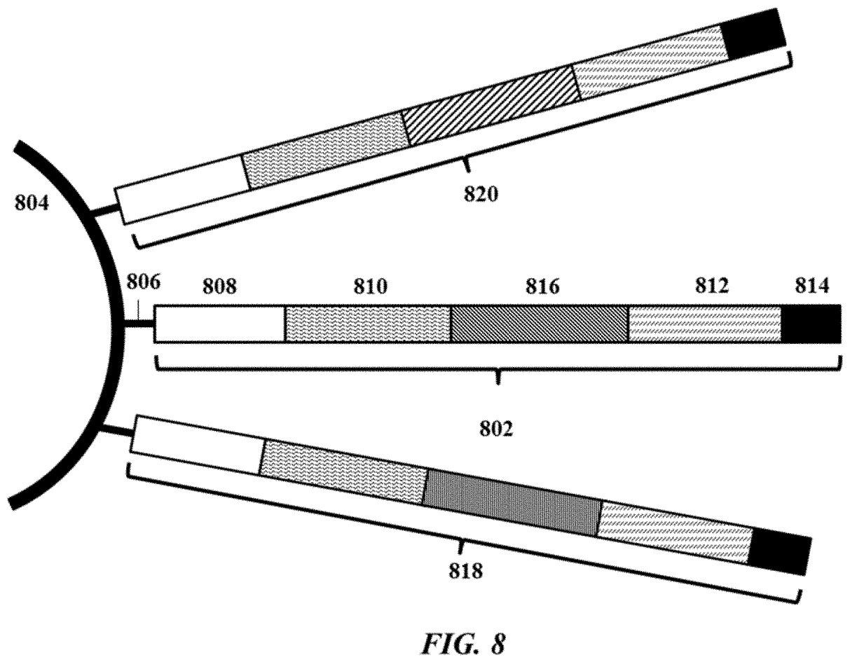

FIG. 8 illustrates an example of a barcode carrying bead. A nucleic acid molecule 802, such as an oligonucleotide, can be coupled to a bead 804 by a releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid molecules (e.g., including nucleic acid molecule 802) coupled to the same bead 804. Alternatively or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

In operation, a biological particle or analyte carrier (e.g., cell, DNA, RNA, etc.) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the released nucleic acid molecules (e.g., 802) can hybridize to the poly-A tail of a mRNA molecule. Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle or analyte carrier (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it can be useful to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, reducing agent free (or DTT free) enzyme preparations may be provided in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, multiply occupied partitions can be provided, e.g., containing two, three, four or more cells and/or microcapsules (e.g., beads) comprising barcoded nucleic acid molecules (e.g., oligonucleotides) within a single partition. Accordingly, as noted above, the flow characteristics of the biological particle or analyte carrier and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of biological particles or analyte carriers (e.g., one biological particle or analyte carrier and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles or analyte carriers and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

### Reagents

In accordance with certain aspects, biological particles or analyte carriers may be partitioned along with lysis reagents in order to release the contents of the biological particles or analyte carriers within the partition. In such cases, the lysis agents can be contacted with the biological particle or analyte carrier suspension concurrently with, or immediately prior to, the introduction of the biological particles or analyte carriers into the partitioning junction/droplet generation zone (e.g., junction 210), such as through an additional channel or channels upstream of the channel junction. In accordance with other aspects, additionally or alternatively, biological particles or analyte carriers may be partitioned along with other reagents, as will be described further below.

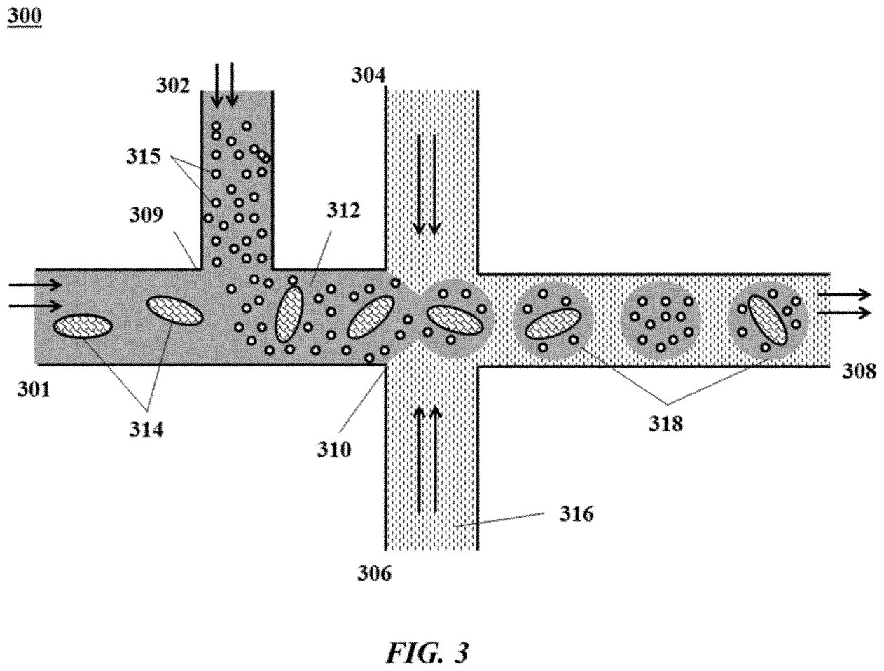

FIG. 3 shows an example of a microfluidic channel structure 300 for co-partitioning biological particles or analyte carriers and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of biological particles or analyte carriers 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of biological particles or analyte carriers 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles or analyte carriers 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual biological particle or analyte carrier 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no biological particles or analyte carriers).

Beneficially, when lysis reagents and biological particles or analyte carriers are co-partitioned, the lysis reagents can facilitate the release of the contents of the biological particles or analyte carriers within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles or analyte carriers that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles or analyte carriers to cause the release of the biological particles or analyte carriers' contents into the partitions. For example, in some cases, surfactant-based lysis solutions may be used to lyse cells. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles or analyte carriers that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles or analyte carriers described above, other reagents can also be co-partitioned with the biological particles or analyte carriers, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles or analyte carriers, the biological particles or analyte carriers may be exposed to an appropriate stimulus to release the biological particles or analyte carriers or their contents from a co-partitioned microcapsule. For example, in some cases, a chemical stimulus may be co-partitioned along with an encapsulated biological particle or analyte carrier to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle or analyte carrier to be released into a partition at a different time from the release of nucleic acid molecules into the same partition.

Additional reagents may also be co-partitioned with the biological particles or analyte carriers, such as endonucleases to fragment a biological particle's or analyte carrier's DNA, DNA polymerase enzymes and dNTPs used to amplify the biological particle's or analyte carrier's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, or analyte carriers such as RNA, DNA, or proteins) contained therein may be further processed within the partitions. In accordance with the methods and systems described herein, the macromolecular component contents of individual biological particles or analyte carriers can be provided with unique identifiers such that, upon characterization of those macromolecular components they may be attributed as having been derived from the same biological particle or particles or analyte carrier or carriers. The ability to attribute characteristics to individual biological particles or analyte carrier or groups of biological particles or analyte carriers is provided by the assignment of unique identifiers specifically to an individual biological particle or analyte carrier or groups of biological particles or analyte carriers. Unique identifiers, e.g., in the form of nucleic acid barcodes can be assigned or associated with individual biological particles or analyte carriers or populations of biological particles or analyte carriers, in order to tag or label the biological particle's or analyte carrier's macromolecular components (and as a result, its characteristics) with the unique identifiers. These unique identifiers can then be used to attribute the biological particle's or analyte carrier's components and characteristics to an individual biological particle or analyte carrier or group of biological particles or analyte carriers.

In some aspects, this is performed by co-partitioning the individual biological particle or analyte carrier or groups of biological particles or analyte carriers with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle or analyte carrier, or to other components of the biological particle or analyte carrier, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles or analyte carriers. These sequences include, e.g., targeted or random/universal amplification primer sequences for amplifying the genomic DNA from the individual biological particles or analyte carriers within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, multiple different barcodes are incorporated within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles or analyte carriers, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

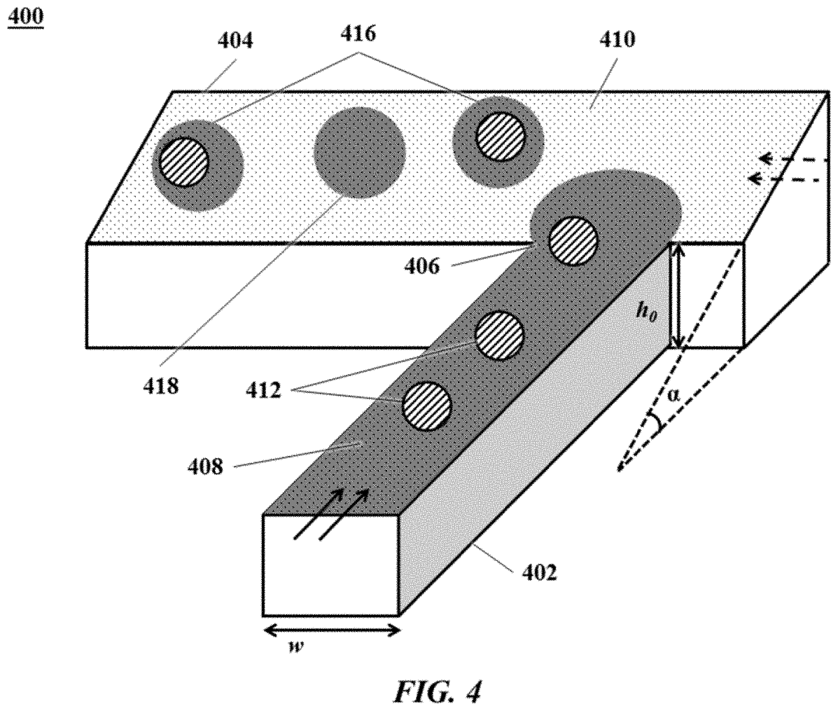

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, a, etc.) of the channel structure 400. A plurality of droplets can be collected in the reservoir 404 by continuously injecting the aqueous fluid 408 from the channel segment 402 through the junction 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more biological particles or analyte carriers, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise biological particles or analyte carriers (e.g., described with reference to FIGS. 1 and 2). In some instances, the aqueous fluid 408 can have a substantially uniform concentration or frequency of biological particles or analyte carriers. As with the beads, the biological particles or analyte carriers can be introduced into the channel segment 402 from a separate channel. The frequency or concentration of the biological particles or analyte carriers in the aqueous fluid 408 in the channel segment 402 may be controlled by controlling the frequency in which the biological particles or analyte carriers are introduced into the channel segment 402 and/or the relative flow rates of the fluids in the channel segment 402 and the separate channel. In some instances, the biological particles or analyte carriers can be introduced into the channel segment 402 from a plurality of different channels, and the frequency controlled accordingly. In some instances, a first separate channel can introduce beads and a second separate channel can introduce biological particles or analyte carriers into the channel segment 402. The first separate channel introducing the beads may be upstream or downstream of the second separate channel introducing the biological particles or analyte carriers.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the junction 406 may have certain geometric features that at least partly determine the sizes of the droplets formed by the channel structure 400. The channel segment 402 can have a height, h₀ and width, w, at or near the junction 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the junction 406 can be inclined at an expansion angle, a. The expansion angle, a, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and a:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, a, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85° or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 408 entering the junction 406 can be between about 0.04 microliters (μL)/minute (min) and about 40 μL/min. In some instances, the flow rate of the aqueous fluid 408 entering the junction 406 can be between about 0.01 microliters (μL)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

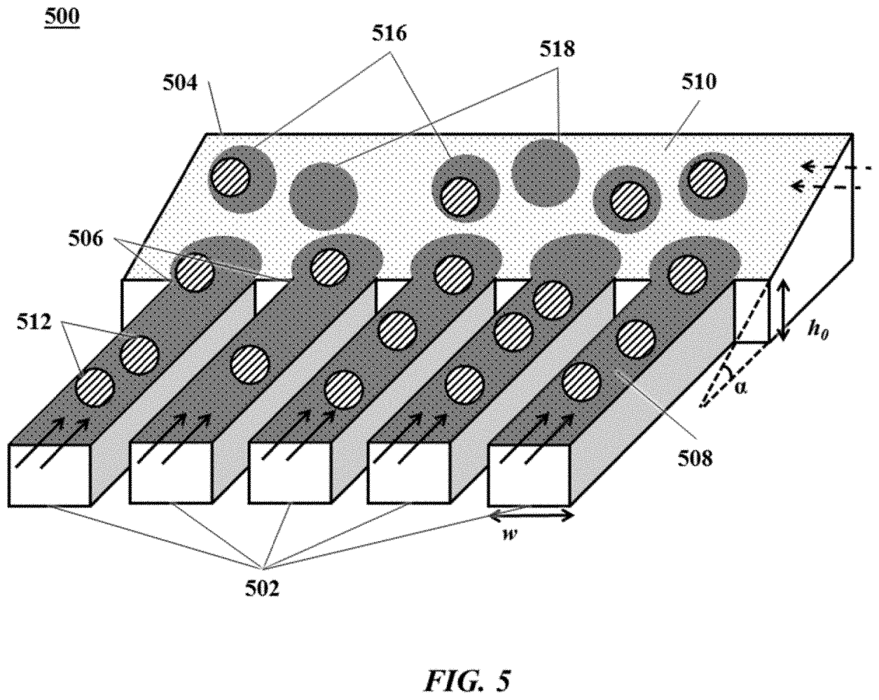

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctions. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the junction where the aqueous fluid 508 and the second fluid 510 meet, droplets can form based on factors such as the hydrodynamic forces at the junction, flow rates of the two fluids 508, 510, fluid properties, and certain geometric parameters (e.g., w, h₀, a, etc.) of the channel structure 500, as described elsewhere herein. A plurality of droplets can be collected in the reservoir 504 by continuously injecting the aqueous fluid 508 from the plurality of channel segments 502 through the plurality of junctions 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and a, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, the geometric parameters for the plurality of channel segments 502 may be varied accordingly to provide a different distribution of droplet sizes.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

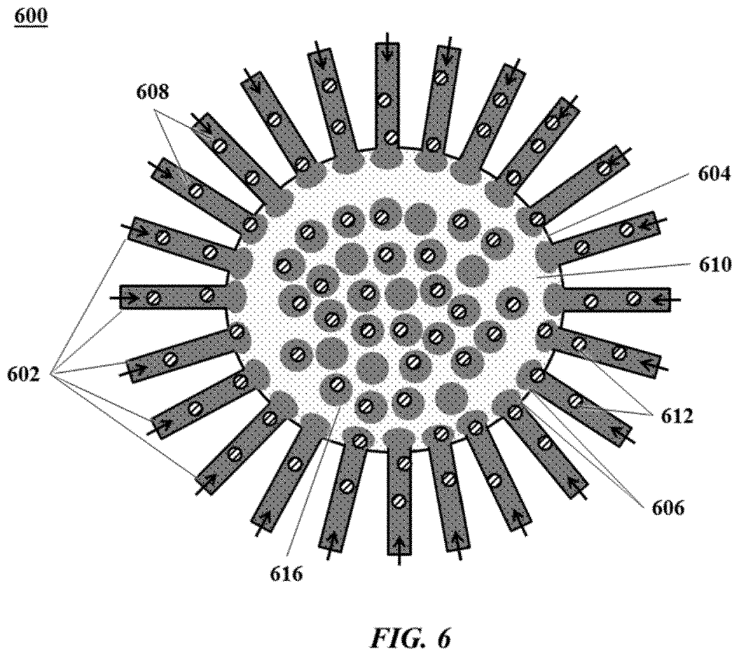

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 2 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctions. Alternatively, the second fluid 610 may be subjected and/or directed to flow in or out of the reservoir 604. For example, the reservoir 604 can be a channel directing the second fluid 610 from upstream to downstream, transporting the generated droplets.

In operation, the aqueous fluid 608 that includes suspended beads 612 may be transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the junction where the aqueous fluid 608 and the second fluid 610 meet, droplets can form based on factors such as the hydrodynamic forces at the junction, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctions 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, a (not shown in FIG. 6) at or near each channel junction. Each channel segment of the plurality of channel segments 602 may have a width, w, and a height, h₀, at or near the channel junction. The geometric parameters, w, h₀, and a, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, the geometric parameters for the plurality of channel segments 602 may be varied accordingly to generate a different distribution of droplet sizes.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle or analyte carrier injected into the droplets may or may not have uniform size.

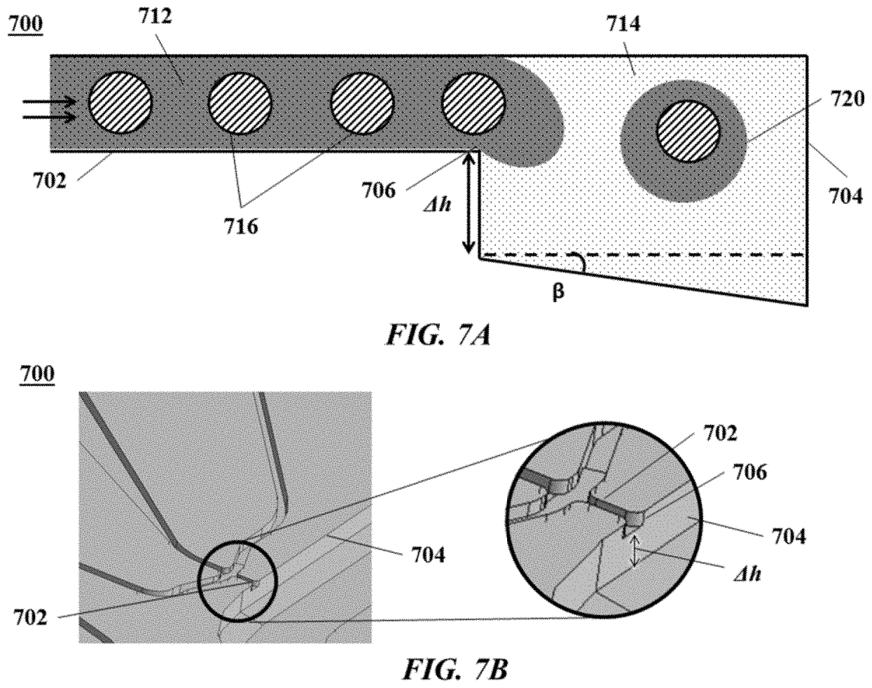

FIG. 7A shows a cross-section view of another example of a microfluidic channel structure with a geometric feature for controlled partitioning. A channel structure 700 can include a channel segment 702 communicating at a channel junction 706 (or intersection) with a reservoir 704. In some instances, the channel structure 700 and one or more of its components can correspond to the channel structure 100 and one or more of its components. FIG. 7B shows a perspective view of the channel structure 700 of FIG. 7A.

An aqueous fluid 712 comprising a plurality of particles 716 may be transported along the channel segment 702 into the junction 706 to meet a second fluid 714 (e.g., oil, etc.) that is immiscible with the aqueous fluid 712 in the reservoir 704 to create droplets 720 of the aqueous fluid 712 flowing into the reservoir 704. At the junction 706 where the aqueous fluid 712 and the second fluid 714 meet, droplets can form based on factors such as the hydrodynamic forces at the junction 706, relative flow rates of the two fluids 712, 714, fluid properties, and certain geometric parameters (e.g., Δh, etc.) of the channel structure 700. A plurality of droplets can be collected in the reservoir 704 by continuously injecting the aqueous fluid 712 from the channel segment 702 at the junction 706.

A discrete droplet generated may comprise one or more particles of the plurality of particles 716. As described elsewhere herein, a particle may be any particle, such as a bead, cell bead, gel bead, biological particle or analyte carrier, macromolecular constituents of biological particle or analyte carrier, or other particles. Alternatively, a discrete droplet generated may not include any particles.

In some instances, the aqueous fluid 712 can have a substantially uniform concentration or frequency of particles 716. As described elsewhere herein (e.g., with reference to FIG. 4), the particles 716 (e.g., beads) can be introduced into the channel segment 702 from a separate channel (not shown in FIG. 7). The frequency of particles 716 in the channel segment 702 may be controlled by controlling the frequency in which the particles 716 are introduced into the channel segment 702 and/or the relative flow rates of the fluids in the channel segment 702 and the separate channel. In some instances, the particles 716 can be introduced into the channel segment 702 from a plurality of different channels, and the frequency controlled accordingly. In some instances, different particles may be introduced via separate channels. For example, a first separate channel can introduce beads and a second separate channel can introduce biological particles or analyte carriers into the channel segment 702. The first separate channel introducing the beads may be upstream or downstream of the second separate channel introducing the biological particles or analyte carriers.

In some instances, the second fluid 714 may not be subjected to and/or directed to any flow in or out of the reservoir 704. For example, the second fluid 714 may be substantially stationary in the reservoir 704. In some instances, the second fluid 714 may be subjected to flow within the reservoir 704, but not in or out of the reservoir 704, such as via application of pressure to the reservoir 704 and/or as affected by the incoming flow of the aqueous fluid 712 at the junction 706. Alternatively, the second fluid 714 may be subjected and/or directed to flow in or out of the reservoir 704. For example, the reservoir 704 can be a channel directing the second fluid 714 from upstream to downstream, transporting the generated droplets.

The channel structure 700 at or near the junction 706 may have certain geometric features that at least partly determine the sizes and/or shapes of the droplets formed by the channel structure 700. The channel segment 702 can have a first cross-section height, h₁, and the reservoir 704 can have a second cross-section height, h₂. The first cross-section height, h₁, and the second cross-section height, h₂, may be different, such that at the junction 706, there is a height difference of Δh. The second cross-section height, h₂, may be greater than the first cross-section height, h₁. In some instances, the reservoir may thereafter gradually increase in cross-section height, for example, the more distant it is from the junction 706. In some instances, the cross-section height of the reservoir may increase in accordance with expansion angle, β, at or near the junction 706. The height difference, Δh, and/or expansion angle, β, can allow the tongue (portion of the aqueous fluid 712 leaving channel segment 702 at junction 706 and entering the reservoir 704 before droplet formation) to increase in depth and facilitate decrease in curvature of the intermediately formed droplet. For example, droplet size may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, β, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

In some instances, the flow rate of the aqueous fluid 712 entering the junction 706 can be between about 0.04 microliters (μL)/minute (min) and about 40 μL/min. In some instances, the flow rate of the aqueous fluid 712 entering the junction 706 can be between about 0.01 microliters (μL)/minute (min) and about 100 μL/min. Alternatively, the flow rate of the aqueous fluid 712 entering the junction 706 can be less than about 0.01 μL/min. Alternatively, the flow rate of the aqueous fluid 712 entering the junction 706 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 712 entering the junction 706. The second fluid 714 may be stationary, or substantially stationary, in the reservoir 704. Alternatively, the second fluid 714 may be flowing, such as at the above flow rates described for the aqueous fluid 712.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

While FIGS. 7A and 7B illustrate the height difference, Δh, being abrupt at the junction 706 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the junction 706, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIGS. 7A and 7B illustrate the expanding reservoir cross-section height as linear (e.g., constant expansion angle, β), the cross-section height may expand non-linearly. For example, the reservoir may be defined at least partially by a dome-like (e.g., hemispherical) shape having variable expansion angles. The cross-section height may expand in any shape.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or analyte carrier or organism types within a population of biological particles or analyte carriers, including, for example, microbiome analysis and characterization, environmental testing, food safety testing, epidemiological analysis, e.g., in tracing contamination or the like.

### Computer Systems

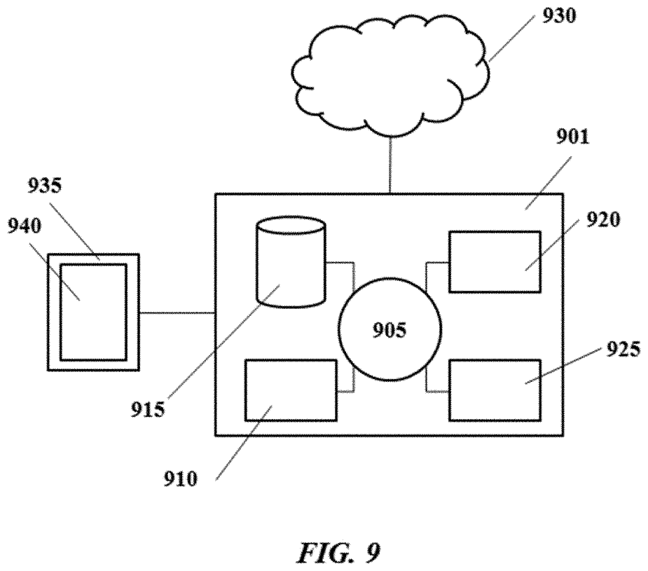

The present disclosure provides computer systems that are programmed to implement methods of the disclosure. FIG. 9 shows an example computer system 901 that can be programmed or otherwise configured to, for example, process and/or analyze a metabolite, control addition of reagents to reaction mixtures, control partition generation, control of reagent addition to partitions, provide conditions sufficient to conduct reactions, obtain and process sequencing data, output sequencing results to a user, provide an interface for user input to control devices coupled to the computer processor. The computer system 901 can regulate various aspects of the present disclosure, such as, for example, regulating fluid flow, delivery of reagents, partition generation, modulate reactions conditions, etc. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, monitoring of sample preparation, monitoring of reactions and/or reaction conditions, monitoring of sequencing, results of sequencing, and permitting user inputs for sample preparation, reactions, sequencing and/or sequencing analysis. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, implement sample preparation protocols, reaction protocols, sequencing protocols, data analysis protocols and system or device operation protocols.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a cell. For example, a biological particle or analyte carrier (e.g., a cell or cell bead) is partitioned in a partition (e.g., droplet), and multiple analytes from the biological particle or analyte carrier are processed for subsequent processing. The multiple analytes may be from the cell. This may enable, for example, simultaneous proteomic, transcriptomic and genomic analysis of the cell.

### EXAMPLES

### Example 1: GEM Generation and Barcoding

A suspension of cells is prepared, where the viability, as measured by trypan blue staining, is greater than 80%. The suspension of cells can be optionally filtered through a 40 μm strainer prior to loading onto a microfluidic chip. A master mix is then prepared which contains the reverse transcriptase (RT) reagent mix, RT primer, DTT, the RT enzyme mix and a volume of cells according to an expected cell recovery number, up to a volume of 100 μL. The master mix may contain riboswitches which may bind to a particular metabolite from a cell and expose a capture sequence on the riboswitch. 90 μL of the master mix is loaded into a first channel of the microfluidic chip. 40 μL of gel beads comprising barcode sequences (e.g., each bead comprising a single barcode sequence) is loaded into a second channel of this chip. 270 μL of partitioning oil is loaded into a third channel of the chip. To generate a gel bead in emulsions (GEMs) comprising a cell and a barcoded gel bead, a specially designed gasket is attached to the chip and then the loaded chip is transferred to a controller. The instrument will recognize the chip and the user can initialize the generation of GEMs by pressing a button. A given GEM may contain one or more sequences for analysis, including, for example, RNA from a cell in the GEM (e.g., mRNA) and/or a riboswitch sequence (e.g., a riboswitch capture sequence) in the GEM. After the instrument has generated the GEMs, 100 μl of GEMs are aspirated from the recovery wells and are dispensed carefully into PCR tube(s).

A gel bead in emulsion-reverse transcriptase (GEM-RT) reaction can then be performed. The tube(s) are loaded into a thermocycler with an incubation of the tube at 53° C. for 45 mins followed by 85° C. for 5 mins and with a reaction volume of 125 μL. After the incubation, the tube(s) can be store at 4° C. or subjected to additional reactions. After thermocycling, a given GEM may contain barcoded cDNA generated from analyte sequences in the GEM, including cDNA corresponding to cellular RNA or a riboswitch sequence, as is discussed above. Barcodes are available from those coupled to or released from a gel bead in the GEM. Barcoding of analytes, including cellular RNA and riboswitch sequences, may proceed as discussed elsewhere herein, and, in some cases with the aid of a template switching reaction.

### Example 2: Post GEM-RT Clean-Up and cDNA Amplification

After generating barcoded nucleic acids in example 1, a recovery reaction is performed. 125 μL of perfluorooctanol, a type of recovery agent, is added to each tube containing the products obtained in Example 1. The perfluorooctanol is allowed to interact with the GEMs for 1 minute without disturbances such as pipetting or vortexing. 125 μL of the recovery agent and oil is slowly removed from the bottom of the tube and discarded. A silane bead clean up mix is prepared by, for each sample, mixing 4 μL of DynaBead MyOne Silane, 5 μL of 1M DTT, 182 μL of a sample clean up buffer, and 9 μL of nuclease-free water. 200 μL of silane clean up mix is added to each sample, mixed well, and then is incubated at room temperature for 10 min. The samples are then placed in a magnetic separator. The supernatant is allowed to clarify, and then the supernatant can be removed and discarded. 300 μL of freshly prepared 80% ethanol is added to each sample while the samples are still placed in the magnetic separator. The ethanol mixture is allowed to let stand for 30 seconds and then is removed and discarded. 200 μL of 80% ethanol is added and again is allowed to let stand for 30 second prior to removal and discard. The samples are then centrifuged and then are returned to the magnetic separator. Any remaining ethanol in the sample is discarded. The samples are allowed to air dry for 1 minute. The samples are then moved from the magnetic separator and 35.5 μL of an elution solution is added. The samples are mixed well and incubated for 1 minute at room temperature. The samples are placed back into the magnetic separator and the solution is allowed to clarify. 35 μL of the supernatant containing the purified GEM-RT product is transferred to a new tube.

To amplify this purified cDNA, a cDNA amplification reaction is performed. This cDNA amplification reaction is performed by adding 50 μL of an amplification master mix (which may contain enzymes, and nucleotides), 5 μL of a cDNA amplification additive mix, 2 μL of a cDNA primer mix, and 8 μL of nuclease free water to the 35 μL of the purified cDNA obtained from the GEM-RT reaction. The cDNA amplification reaction is mixed well, centrifuged briefly, capped, and then loaded into a thermocycler. The following program is run with a lid temperature of 105° C. with a total reaction volume of 100

| Step | Temperature | Time |
|---|---|---|
| 1 | 98° C. | 3:00 |
| 2 | 98° C. | 0:15 |
| 3 | 67° C. | 0:20 |
| 4 | 72° C. | 1:00 |

| | |
|---|---|
| 5 | Go to step 2, 8-14 cycles |

| | | |
|---|---|---|
| 6 | 72° C. | 1:00 |
| 7 | 4° C. | Hold |

Once the cDNA is amplified, the cDNA corresponding to for example, riboswitches sequences or cellular RNA can be isolated or purified by using SPRIselect Reagent to size select for the appropriate fragments size corresponding to a desired cDNA. After cDNA amplification, 60 μL of SPRIselect Reagent is added to each sample to achieve a 0.6× ratio. The samples are then mixed well, incubated at room temperature for 5 min, and then placed into a magnetic separator. Once the solutions are substantially clear, 150 μL of supernatants are collected in separate tubes and stored at 4° C. The supernatant of this size selection reaction will contain sequences of sizes corresponding to riboswitch sequences. The pellets are then washed twice with 200 μL of 80% ethanol per wash and let air dry for 2 min. The samples are then removed from the magnetic separator and 40.5 μL of elution buffer is added to each sample. With a pipette, each sample is mixed and then incubated at room temperature for 2 min. The samples are placed back on the magnetic separator for 4 mins and the solution is allowed to clarify. 40 μL of each sample is then transferred to a new tube for downstream processing

### Example 3: Cellular RNA Library Selection and Preparation

cDNA corresponding to cellular RNA, for example, mRNA, can be selected and then prepared for downstream sequencing reactions. This cDNA can be generated by use of materials and protocols discussed in Example 1, followed by cDNA amplification as discussed in Example 2. For example, cDNA corresponding to gene expression may be generated by GEMs comprising gel beads comprising capture sequences for mRNA. The cDNA can be size selected by the use of the SPRIselect Reagent, such that cDNA not pertaining to cellular RNA can be separated (e.g. riboswitch related cDNA) from the cDNA pertaining to cellular RNA, as mentioned in Example 2. These cDNA can then be prepared for downstream sequencing reactions by a process of fragmentation, end repair and A-tailing the cDNA. To perform the fragmentation, end repair and A-tailing, a thermocycler can be prepared with the following incubation protocol:

| Lid Temperature | Reaction Volume | Run Time |
|---|---|---|
| 65° C. | 50 μL | 35 min |

| Step | Temperature | Time |
|---|---|---|
| Pre-cool block | 4° C. | Hold |
| Fragmentation | 32° C. | 5:00 |
| End Repair & A-tailing | 65° C. | 30:00 |
| Hold | 4° C. | Hold |

The protocol is started, awaiting initiation by the user to continue past the first pre-cooling step. A fragmentation mix is then prepared using, per reaction, 5 μL of fragmentation buffer and 10 μL of a fragmentation enzyme blend. 15 μL of this fragmentation mix is dispensed in each well of a PCR 8-tube strip which has been placed on an ice block. 17.5 μL of purified, size selected cDNA corresponding to cellular RNA and 17.5 μL of nuclease-free water are added to each well of the PCR tube strip. The solution is mixed well and then placed in the thermocycler and thermocycler program is allowed to continue to the Fragmentation step by the users' prompt. After the thermocycler protocol is completed, 30 μL of SPRIselect Reagent is added to each sample, achieving a 0.6× ratio. The samples are mixed well, incubated at room temperature for 5 min, and then placed in a magnetic separator. Once the solution is clear, the supernatants are removed and transferred to a new tube. The tubes with the magnetic bead can be discarded. 10 μL of SPRIselect Reagent is then added to each sample, achieving a 0.8× ratio. The samples are mixed well and incubated at room temperature for 5 min. Following incubation, the samples are placed on the magnetic separator and the solutions are allowed to clarify. The supernatants are then removed and discarded. The pellets are then washed twice with 200 μL of 80% ethanol per wash and then let air dry for 2 min. The sampled are removed from the magnetic separator and 50.5 μL of elution buffer is added and mixed well with a pipette. After the samples are incubated at room temperature for 2 min, the samples are placed back on the magnetic separator for 4 minutes for the solution to clarify. 50 μL of each sample is transferred to a new tube for use in the next step. For the next step, an adaptor ligation mix is prepared using 17.5 μL of nuclease-free water, 20 μL of ligation buffer, 10 μL of DNA ligase, and 2.5 μL of Adaptor Mix. 50 μL of the adaptor ligation mix is added the 50 μL of each sample (which was fragmented, end repaired, and A-tailing size selected) for a total 100 μL volume. The solutions are mixed well and briefly centrifuged before incubating in a thermocycler at 20° C. for 15 mins, with the lid at 30° C. After incubation and the ligation of the adaptors, 80 μL of SPRIselect Reagent is added to each sample to achieve a 0.8× ratio. The samples are then mixed well, incubated at room temperature for 5 min, and then placed into a magnetic separator. Once the solutions are substantially clear, the supernatants are removed and discarded. The pellets are then washed twice with 200 μL of 80% ethanol per wash and let air dry for 2 min. The samples are then removed from the magnetic separator and 30.5 μL of elution buffer is added to each sample. With a pipette, each sample is mixed and then incubated at room temperature for 2 min. The samples are placed back on the magnetic separator for 4 mins and the solution is allowed to clarify. 30 μL of each sample is then transferred to a new tube for processing via sample index PCR. The sample index PCR will add an index to each sample. A sample index PCR (SI-PCR) reaction mix is prepared by mixing 8 μL of nuclease-free water, 50 μL of amplification master mix, and 2 μL of the SI-PCR primer. This 60 μL solution is added to each tube containing the 30 μL of each sample. 10 μL of a sample index is added to the sample. To keep track of the indices, it is highly suggested to record the index assignment for each sample. After the sample is mixed well and briefly centrifuged, each sample is incubated in a hot-lid thermocycler using the following protocol:

| Lid Temperature | Reaction Volume | Run Time |
|---|---|---|
| 105° C. | 100 μL | 25-40 min |

| Step | Temperature | Time |
|---|---|---|
| 1 | 98° C. | 0:45 |
| 2 | 98° C. | 0:20 |
| 3 | 54° C. | 0:30 |
| 4 | 72° C. | 0:20 |

| | |
|---|---|
| 5 | Go to step 2, 10 to 14 cycles, depending on input mass |

| | | |
|---|---|---|
| 6 | 72° C. | 1:00 |
| 7 | 4° C. | Hold |

After incubation in the thermocycler, the sample can be stored at 4° C. overnight or −20° C. until the next step is initiated. To clean up and perform a double size-selection, 60 μL of SPRIselect Reagent is added to each sample, achieving a 0.6× ratio. The sample are then mixed well and incubated at room temperature for 5 min. After incubation, the samples are placed in a magnetic separator until the solution is clear. Each supernatant is then removed and transferred to a new tube. The tubes with the remaining beads can be discarded. 20 μL of SPRIselect Reagent is added to each supernatant in the new tube to achieve a 0.8× ratio. These samples are mixed well and then incubated at room temperature for 5 min. The samples are then placed on the magnetic separator, and the solution is allowed to clarify. The supernatants are then removed and discarded. Using 200 μL of 80% ethanol per wash, the beads are washed twice and then let air dry for 2 mins. The samples are then removed from the magnetic separator and 35.5 μL of elution buffer is added. After pipette mixing and then incubating at room temperature for 2 mins, the samples are placed back on the magnetic separator for 4 min and the solutions are allowed to clarify. 35 μL of each sample is transfer to a new tube for storage at 4° C. overnight or −20° C. for long term storage. Additionally quality control and quantification can be done by running the purified sample on an Agilent Bioanalyzer High Sensitivity Chip for qualitative analysis of the cDNA library and an estimation of concentration and size.

### Example 4: Riboswitch Library Selection and Preparation

A riboswitch library which pertains to certain metabolites found in the cells can be selected and prepared using materials and protocols from Example 1. The GEMs generated as seen in Example 1 contain captures sequences that bind to the riboswitches in the presence of a metabolite, aiding in the determination of the cell metabolome. The cDNA corresponding to these riboswitches can be generated via a reverse-transcriptase reaction as described in Example 1 and amplified and purified as described in Example 2. To clean up and perform a size selection for cDNA corresponding to riboswitches, 60 μL of SPRIselect Reagent is added to each 150 μL of supernatant sample obtained in Example 2, achieving a 1.2× ratio. The samples are mixed well and incubated at room temperature for 5 mins. The samples are then placed in a magnetic separator until the solution is clear. The supernatant is then removed and discarded. With 200 μL of 80% ethanol per wash, the pellets are washed twice and then let air dry for 2 min. The samples are removed from the magnetic separator and 40.5 μL of an elution buffer is added. Using a pipette, the samples are mixed well and incubated at room temperature for 2 min. The samples are then returned to the magnetic separator and allowed to sit for 4 minutes to allow the solution to become clear. 40 μL of the sample is transferred to a new tube. To add indices to samples, a sample index PCR (SI-PCR) mix is prepared by mixing 8 μL of nuclease-free water, 50 μL of amplification mix and 2 μL of SI-PCR primer for each sample. 60 μL of the sample PCR index mix is added to each PCR tube, and 5 μL of purified riboswitch cDNA product is additionally added to each tube. Then, 25 μL of nuclease-free water and 10 μL of sample index are added to each sample. It is recommended that the index assignment is recorded for proper indexing. The samples are mixed well, centrifuged briefly, and then incubated in a hot-lid thermocycler using the following protocol:

| Lid Temperature | Reaction Volume | Run Time |
|---|---|---|
| 105° C. | 100 μL | 25-30 min |

| Step | Temperature | Time |
|---|---|---|
| 1 | 98° C. | 0:45 |
| 2 | 98° C. | 0:20 |
| 3 | 54° C. | 0:30 |
| 4 | 72° C. | 0:20 |

| | |
|---|---|
| 5 | Go to step 2, 6 cycles |

| | | |
|---|---|---|
| 6 | 72° C. | 1:00 |
| 7 | 4° C. | Hold |

The samples after thermocycling can be stored overnight at 4° C. or at −20° C. until purification steps are initiated. To perform cleanup and size-selection, 100 μL of SPRIselect reagent is added to each sample to achieve a 1.0× ratio. The samples are mixed well and then incubated at room temperature for 5 min. The samples are then placed on a magnetic separator and the sample is allowed to clarify. The supernatant are then removed and then discarded. Using 200 μL of 80% ethanol per wash, the beads are washed twice and then let air dry for 2 mins. The samples are then removed from the magnetic separator and 35.5 μL of elution buffer is added. After pipette mixing and then incubating at room temperature for 2 mins, the samples are placed back on the magnetic separator for 4 min and the solutions are allowed to clarify. 35 μL of each sample is transferred to a new tube. 28 μL of SPRIselect reagent is added to each sample, achieving a 0.8× ratio. The samples are then mixed well and incubated for 5 min. before placing the samples in a magnetic separator and letting the solution clarify. The supernatant are then removed and then discarded. Using 200 μL of 80% ethanol per wash, the beads are washed twice and then let air dry for 2 mins. The samples are then removed from the magnetic separator and 35.5 μL of elution buffer is added. After pipette mixing and then incubating at room temperature for 2 mins, the samples are placed back on the magnetic separator for 4 min and the solutions are allowed to clarify. 35 μL of each sample is transferred to a new tube. After incubation in the thermocycler, the sample can be stored at 4 C overnight or −20 C for long term storage. Additionally, quality control and quantification can be done by running the purified sample on an Agilent Bioanalyzer High Sensitivity Chip for qualitative analysis of the cDNA library and an estimation of concentration and size.

### Example 5: Library Sequencing

Cellular RNA and riboswitch libraries generated from Example 3 and 4, respectively, both can contain standard Illumina paired-end constructs which begin and end with P5 and P7. Reads 1 and 2 can use standard Illumina sequencing primer sites used in paired-end sequencing. Read 1 can be used to sequence barcodes and unique molecular identifier. Read 2 can be used to sequence the insert or the riboswitch sequence. An index read of 8 cycles can be optionally but preferentially performed. By way of the above examples, it is possible to purify two distinct libraries. One library may contain gene expression information correlated to the cellular RNA from every single cell successfully captured in partitions (Example 3). The other library, which as discussed in Example 4, is a riboswitch library, may contain metabolome information from every single cell successfully captured in the partitions.

Thus, both gene expression and metabolome information can be traced back to the same cells utilizing the barcoding and indexing described elsewhere herein and then processed accordingly with software tools. The information obtained in these examples and the many embodiments of this disclosure can be of great use in understanding the current state of a cell or population of cells. Gene expression information is highly informative, but as many functions performed by the cells are dictated by the metabolites present, this disclosure may allow an investigator to more thoroughly classify and sub-classify cells based on various molecular patterns.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for processing or analyzing a metabolite from a cell, comprising:
(a) generating a partition comprising (i) said metabolite, (ii) a single bead comprising a plurality of nucleic acid cell barcode molecules each comprising a cell barcode sequence and a capture sequence, which cell barcode sequence uniquely corresponds to said cell, and (iii) a molecular complex that is capable of coupling to said metabolite, wherein said molecular complex comprises a molecular complex capture sequence and a molecular complex identifier sequence, wherein said molecular complex capture sequence is inaccessible to said capture sequence in the absence of said metabolite coupled to said molecular complex;
(b) in said partition, providing conditions sufficient for said metabolite to couple to said molecular complex to (i) render said molecular complex capture sequence accessible to said capture sequence of a given nucleic acid cell barcode molecule of said plurality of nucleic acid cell barcode molecules and (ii) permit said capture sequence to couple to said molecular complex capture sequence; and
(c) with said capture sequence coupled to said molecular complex capture sequence, using said given nucleic acid cell barcode molecule and said molecular complex to synthesize a nucleic acid molecule comprising (1) a first nucleic acid sequence corresponding to said molecular complex identifier sequence, and (2) a second nucleic acid sequence corresponding to said cell barcode sequence, wherein said first nucleic acid sequence and said second nucleic acid sequence permit said metabolite to be identified as corresponding to said cell.

2.-31. (canceled)
